(19) United States
(12) Patent Application Publication
(10) Pub. No.: US 2018/0235506 A1
(43) Pub. Date: Aug. 23, 2018

(54) METHOD AND DEVICE FOR MEASURING BIOMETRIC DATA USING UWB RADAR

(71) Applicant: WRT LAB CO., LTD., Seoul (KR)

(72) Inventors: Sung Ho CHO, Seoul (KR); Faheem KHAN, Seoul (KR); Jeong Woo CHOI, Seoul (KR)

(21) Appl. No.: 15/957,941

(22) Filed: Apr. 20, 2018

Related U.S. Application Data
(63) Continuation of application No. 14/748,061, filed on Jun. 23, 2015.

(30) Foreign Application Priority Data
Sep. 26, 2014 (KR) 10-2014-0129383
Jun. 17, 2015 (KR) 10-2015-0085689

(51) Int. Cl. A61B 5/05; A61B 5/113; A61B 5/00; A61B 5/024

(52) U.S. Cl. CPC A61B 5/0507; A61B 5/1135; A61B 5/024; A61B 5/7278

(57) ABSTRACT

A method and device for measuring biometric data using a UWB radar are disclosed. The disclosed method includes: (a) determining a respiratory rate by using reflection signals reflected off a target patient; (b) extracting respiration signals from the reflection signals, removing the extracted respiration signals from the reflection signals and converting into signals of a frequency domain; and (c) determining a heartbeat rate by detecting peak values from the frequency-domain signals converted in said step (b).

### CROSS-REFERENCE TO RELATED APPLICATIONS

This application is a continuation of a U.S. patent application Ser. No. 14/748,061, filed on Jun. 23, 2015, which claims the benefit of Korean Patent Application No. 10-2014-0129383, filed with the Korean Intellectual Property Office on Sep. 26, 2014, the disclosure of which is incorporated herein by reference in its entirety.

### BACKGROUND

### 1. Technical Field

The present invention relates to a method and device for measuring biometric data, more particularly to a method and device for measuring biometric data using a UWB radar.

### 2. Description of the Related Art

UWB refers to radio technology which uses a frequency band of 500 MHz or more or in which the value defined for the fractional bandwidth is 25% or higher. The fractional bandwidth represents the bandwidth of a signal in relation to the center frequency. That is, UWB is a radio technique that uses the frequencies of a wide band and provides various advantages such as high range resolution, penetrability, strong immunity to narrowband noise, and compatibility with other devices that share frequencies.

The IR-UWB (impulse-radio ultra-wideband) radar (hereinafter referred to as ‘UWB radar’) technology, which merges such UWB technology with radar, entails recognizing the surrounding environment by transmitting impulse signals of very short duration that have wideband properties in a frequency region and then receiving the signals that are reflected off objects and people.

In a UWB radar system, impulse signals having a duration between several nanoseconds to several picoseconds are generated at a signal generator unit and then emitted at a wide angle or an angle of a narrow band through a transmitter antenna. The emitted signals may be reflected off various objects or people in the environment, and the reflected signals may proceed through a receiver antenna and an ADC to be converted into digital signals.

Because of the advantages of the UWB radar described above, research on utilizing the UWB radar is being conducted in numerous fields. Research for technology development is currently under way in various areas such as medical devices for measuring the respiratory rate and heartbeat rate, portable radar devices for rescue operations at disaster sites, people-counting devices for counting the number of people in a certain area, and the like. One such example is Korean Patent Publication No. 10-2013-0020835 (published Sep. 4, 2014) entitled “UWB-based contactless biometric signals tester”, which discloses a method of providing a remote health care service by means of biometric signals measured using a UWB radar.

However, since the amount of movement resulting from heartbeat is significantly smaller compared to the amount of movement resulting from a person's breathing, there is much difficulty in measuring heartbeat rates.

### SUMMARY

An aspect of the invention is to provide a method and device for measuring biometric data by using a UWB radar.

In particular, an aspect of the invention aims to measure a target patient's heartbeat rate accurately by using a UWB radar.

To achieve the objective above, an embodiment of the invention provides a method of measuring biometric data using a UWB radar that includes: (a) determining a respiratory rate by using reflection signals reflected off a target patient; (b) extracting respiration signals from the reflection signals, removing the extracted respiration signals from the reflection signals and converting into signals of a frequency domain; and (c) determining a heartbeat rate by detecting peak values from the frequency-domain signals converted in said step (b).

Extracting the respiration signals in said step (b) can include: (b1) calculating a mean value of magnitude values of sample times within a second reference time from a particular sample time excluding sample times within a first reference time from the particular sample time, the second reference time being longer than the first reference time; and (b2) replacing a magnitude value of the particular sample time with the mean value, wherein said steps (b1) and (b2) are performed for all sample times in a predesignated segment.

Step (c) can include determining the heartbeat rate as a frequency corresponding to a peak value within a predesignated frequency range from among a plurality of detected peak values.

Step (c) can include removing the harmonic components of the respiratory rate; and determining the heartbeat rate by using the peak values from which the harmonic component have been removed.

Steps (a) through (c) can be performed multiple times, and step (c) can include determining the heartbeat rate by using at least one of a repetition frequency of peak values and a magnitude of a peak values.

Removing the harmonic components can include detecting multiple peak values within a predesignated frequency range and removing the peak values corresponding to harmonic components from among the detected peak values, where the predesignated frequency range can be determined based on the heartbeat rate of the target patient.

Another aspect of the invention provides a device for measuring biometric data using a UWB radar that includes: a frequency converter unit configured to convert reflection signals reflected off a target patient into frequency-domain signals; a respiratory rate determiner unit configured to determine a respiratory rate by using the frequency-domain signals for the reflection signals converted by the frequency converter unit; a peak detector unit configured to detect a plurality of peak values from frequency-domain signals obtained by extracting respiration signals from the reflection signals and removing the extracted respiration signals from the reflection signals and to remove harmonic components of the determined respiratory rate; and a heartbeat rate determiner unit configured to determine a heartbeat rate as one of the peak values remaining after removing the harmonic components.

The respiration signals are extracted by performing a procedure for all sample times within a predesignated segment, the procedure comprising: calculating a mean value of magnitude values of sample times within a second reference time from a particular sample time excluding sample times within a first reference time from the particular sample time, the second reference time being longer than the first reference time; and replacing a magnitude value of the particular sample time with the mean value.

The heartbeat rate determiner unit can determine the heartbeat rate as a frequency corresponding to a peak value within a predesignated frequency range from among the peak values detected. The heartbeat rate determiner unit can determine the heartbeat rate using at least one of a repetition frequency of the peak values and a magnitude of the peak values for the reflection signals obtained over multiple repetitions.

Still another aspect of the invention provides a method of measuring biometric data using a UWB radar that includes: analyzing frequency components after obtaining frequency-domain signals for reflection signals reflected off a target patient; repeating the analyzing for a predesignated number of times to detect multiple peak values in a predesignated frequency range and removing the harmonic components for a maximum peak value from the frequency components; and determining a heartbeat rate by using at least one of a repetition frequency and a magnitude of the peak values from among frequencies for the peak values from which the harmonic components have been removed.

Yet another aspect of the invention provides a device for measuring biometric data using a UWB radar that includes: a frequency converter unit configured to convert reflection signals reflected off a target patient into frequency-domain signals and analyze frequency components; a respiratory rate determiner unit configured to determine a respiratory rate by using the frequency-domain signals for the reflection signals converted by the frequency converter unit; a peak detector unit configured to detect multiple peak values from the results of repeating the frequency component analysis a predesignated number of times and removing harmonic components of the respiratory rate frequency determined by the respiratory rate determiner unit; and a heartbeat rate determiner unit configured to determine a heartbeat rate by using at least one of a repetition frequency and a magnitude of the peak values from among the frequencies for peak values from which the harmonic components have been removed.

Certain embodiments of the invention provide the advantage of accurately determining the heartbeat rate by suitably detecting signals resulting from heartbeats which are relatively weaker in signal magnitude.

Also, certain embodiments of the invention provide the advantage of increased accuracy by detecting the frequency components that correspond to heartbeats and excluding the harmonic components of frequency components that correspond to the target patient's respiration.

Also, an embodiment of the invention may further improve accuracy by repeatedly measuring a target patient and determining the heartbeat rate using at least one of the repetition frequency and magnitude of a peak value from the frequency for a detected peak value.

Additional aspects and advantages of the present invention will be set forth in part in the description which follows, and in part will be obvious from the description, or may be learned by practice of the invention.

### BRIEF DESCRIPTION OF THE DRAWINGS

FIG. 1 shows a frequency spectrum associated with a method of measuring biometric data according to an embodiment of the invention.

FIG. 2 illustrates a method of measuring biometric data according to an embodiment of the invention.

FIG. 3 illustrates a method of measuring biometric data according to another embodiment of the invention.

FIG. 4 illustrates the extraction of respiration signals from the received reflection signals according to an embodiment of the invention.

FIG. 5 shows graphs representing a waveform for the reflections signals of a UWB radar and a waveform for signals from which the respiration signals have been removed.

FIG. 6 is a graph illustrating the signals of the frequency domain when respiration signals have been removed from the received signals.

FIG. 7 is a flowchart illustrating a method of determining a heartbeat rate according to an embodiment of the invention.

FIG. 8 represents the repetition frequencies and mean magnitudes of peak values detected during repetitions of the method of measuring biometric data illustrated in FIG. 7.

FIG. 9 illustrates a device for measuring biometric data according to an embodiment of the invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In describing the drawings, like reference numerals are used for like elements.

Certain embodiments of the invention are described below in more detail with reference to the accompanying drawings.

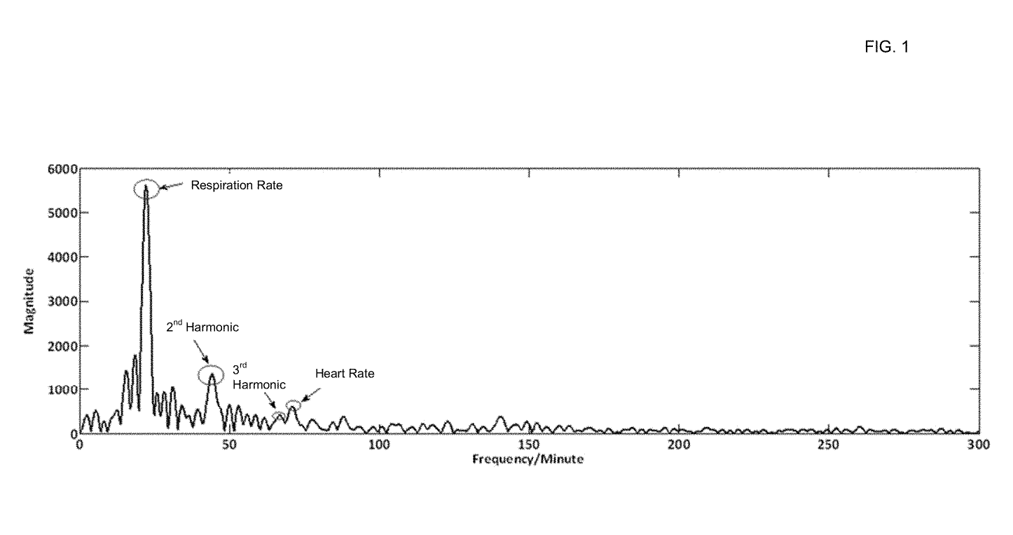

FIG. 1 shows a frequency spectrum associated with a method of measuring biometric data according to an embodiment of the invention.

Respiration and heartbeat cause minute movements in a target patient, and an embodiment of the invention can analyze such movements by using a UWB radar to detect the respiratory rate and heartbeat rate.

By using a UWB radar to transmit radar signals to the target patient, receiving the reflection signals reflected off the target patient, and analyzing the frequency components, it is possible to obtain a spectrum of frequency components, an example of which is shown in FIG. 1. A peak value can be generated at a particular frequency according to the movement of the target patient, while peak values can also be generated by noise and harmonic components. Here, it may be desirable to keep the target patient stationary without any other movements.

When there is no periodical movement other than the movements caused by respiration and heartbeat, the movement resulting from respiration is generally larger than the movement resulting from heartbeat. As such, the frequency representing the maximum peak value in the frequency spectrum may correspond to the respiratory rate. In FIG. 1, the respiratory rate corresponding to the maximum peak value is 22.3 times per minute.

The movement resulting from heartbeat is relatively smaller, and it is not easy to detect heartbeats due to the harmonic component of the frequency component caused by respiration and noise, etc. In particular, the harmonic component may be mixed with exterior noise to have a considerably large value and can pose as an obstacle to detecting the heartbeat rate.

According to an embodiment of the invention, the frequency for the maximum peak value within a predesignated frequency range can be determined as the heartbeat rate. Here, the frequency range can be designated in consideration of typical heartbeat rates for humans. The normal heartbeat rate for a typical person is from 50 to 100 times per minute, and if the frequency range is designated as such, the heartbeat rate can be determined to be 66.7 times per minute as illustrated in FIG. 1.

As the frequency for the maximum peak value is determined as the heartbeat rate in the predesignated frequency range, the second harmonic waves of the frequency component resulting from respiration can be excluded from consideration for the heartbeat rate. However, there may be occasions in which third harmonic waves from the respiratory rate are larger than the heartbeat rate, and thus the signals associated with respiration (particularly the harmonic components of the respiration signals) may affect the identifying of the heartbeat rate. An embodiment of the invention provides a method that can accurately identify the heartbeat rate.

Also, in order to identify the heartbeat rate more accurately, an embodiment of the invention may analyze the frequency component of the target patient over a predesignated number of repetitions. If, for example, the analysis is performed over five repetitions, then five sets of data can be outputted for the frequency spectrum data such as that shown in FIG. 1. For each set of frequency spectrum data, the frequency component of the peak value may be extracted, a total of five candidates may be prepared that include the frequency data for the peak values, and the heartbeat rate of the target patient can be identified by identifying which frequency has the most number of peak values or which frequency has the highest magnitude in the candidates.

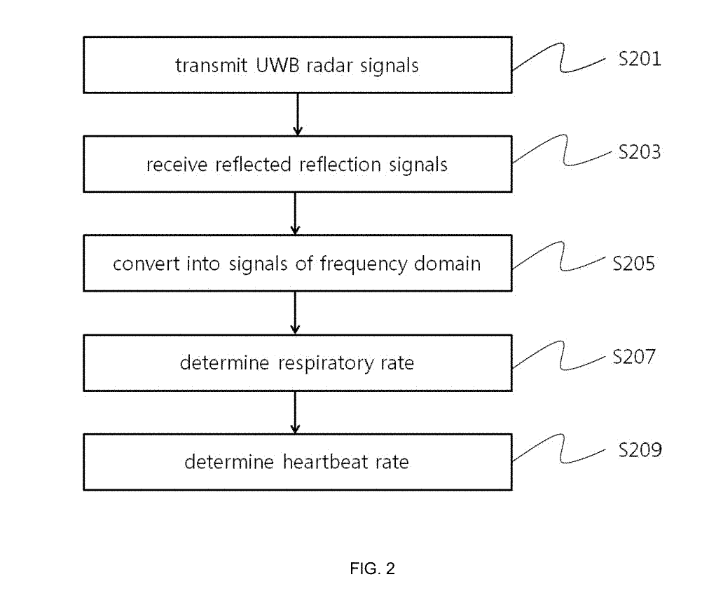

A more detailed description is provided below with reference to the accompanying drawings. FIG. 2 illustrates a method of measuring biometric data according to an embodiment of the invention. The method of measuring biometric data according to an embodiment of the invention can be performed by a device for measuring biometric data that includes a UWB radar.

Referring to FIG. 2, a device for measuring biometric data according to an embodiment of the invention may transmit UWB radar signals to a target patient who is in a stationary position (S201). The UWB radar signals can be impulse signals. The shorter the cycle of the pulses at the UWB radar, the greater is the amount of data that can be included. As defined by the FCC, a UWB radar generates pulse signals having pulse widths of merely several nanoseconds to provide a bandwidth of 500 MHz or greater or provide a bandwidth that is 20% or higher compared to the center frequency.

The device for measuring biometric data according to an embodiment of the invention may receive reflection signals reflected off the target patient (S203) and convert the reflection signals into frequency-domain signals (S205). The conversion into frequency-domain signals can be achieved by way of Fourier transforms.

The device for measuring biometric data according to an embodiment of the invention may analyze the converted frequency signals and determine the respiratory rate as the frequency for the maximum peak value from among the frequency components (S207). As described above, the peak value occurring at a particular frequency as a result of the degree of movement of the target patient can be obtained by way of frequency analysis. The movement by the target patient in a stationary state is the largest during respiration, and as such, the frequency for the maximum peak value can be determined as the respiratory rate.

Also, the device for measuring biometric data according to an embodiment of the invention may determine the heartbeat rate as the frequency for the maximum peak value in a predesignated frequency range associated with the heartbeat rate (S209). The frequency rate can be designated in various ways according to the pertinent circumstances. From among the frequency components, it may be preferable to determine the heartbeat rate as the frequency corresponding to the maximum peak value in a predesignated frequency range, with the harmonic components for the maximum peak value corresponding to the respiratory rate excluded. That is, the harmonic components for the maximum peak value corresponding to the respiratory rate can be ignored, and the heartbeat rate can be determined.

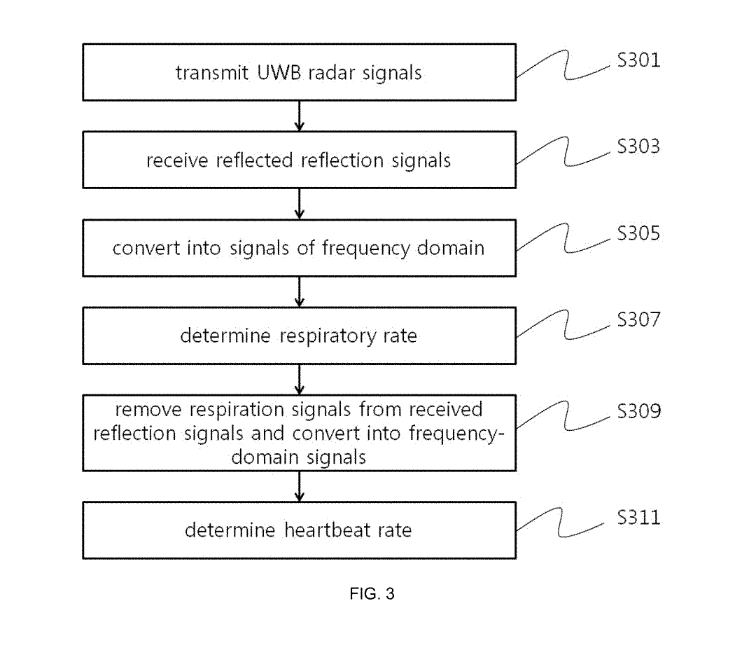

Due to the influence of external noise, etc., there can be occurrences in which a peak value has a greater magnitude than the peak value corresponding to the heartbeat rate within the predesignated frequency range. A method of determining the heartbeat rate more accurately is described below with reference to FIG. 3.

FIG. 3 illustrates a method of measuring biometric data according to another embodiment of the invention.

Compared with the embodiment illustrated in FIG. 2, FIG. 3 additionally includes a process of removing respiration signals from the received reflection signals and converting the reflection signals, from which the respiration signals have been removed, into signals of the frequency domain (S309) after the determining of the respiratory rate from the biometric signals (S307). According to the embodiment illustrated in FIG. 3, the frequency-domain signals are acquired after removing the respiration signals from the received reflection signals, in order to minimize the potential inaccuracy of heartbeat rate detection due to the respiration signals.

The frequency-domain signals from which the respiration signals have been removed may be less affected by the harmonic waves associated with respiration, and the detection of heartbeat rate can be made more accurate compared to the embodiment illustrated in FIG. 2.

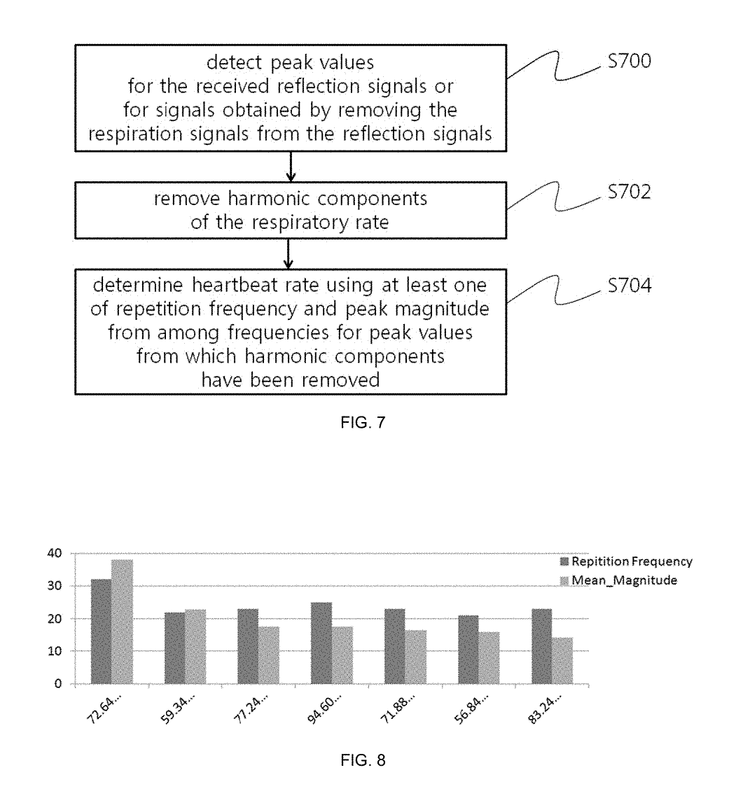

Of course, since the respiration signals cannot be removed entirely, the harmonic components of the respiration signals may still adversely affect accurate heartbeat rate detection. A more detailed method of detecting the heartbeat rate that overcomes this problem will be described later with reference to FIG. 7.

According to the present invention, the respiration signals are extracted from the received reflection signals and then the heartbeat signals are obtained by removing the extracted respiration signals from the received reflection signals.

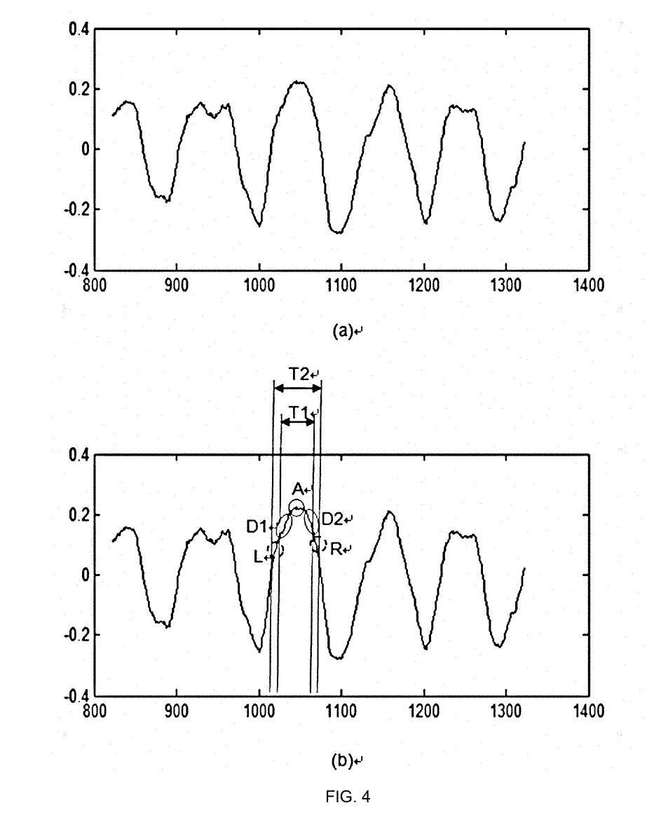

FIG. 4 illustrates the extraction of respiration signals from the received reflection signals according to an embodiment of the invention.

In FIG. 4, the upper diagram shows a graph representing the received reflection signals, while the lower diagram illustrates a method of extracting the respiration signals.

In the upper diagram of FIG. 4, the reflection signals include the respiration signals and heartbeat signals. The respiration signals and the heartbeat signals are cyclical signals and thus have cyclical forms.

While FIG. 4 shows a continuous signal, the signals actually received are discontinuous signals that have a particular magnitude for each sampling time. In an embodiment of the invention, the operation of extracting the respiration signals from the reflection signals may entail replacing the magnitude value of a certain sampling time with another value. Ultimately, this can be defined as an operation of transforming the received reflections signals. The transformation of the received reflection signals can be performed for each associated sampling time and may entail transforming the magnitude value of a particular sampling time into another value.

For extracting the respiration signals from the received reflection signals, two reference times of T1 and T2 may be set. The second reference time T2 may be set larger than the first reference time T1.

Referring to the lower diagram of FIG. 4, the mean magnitude value may be obtained for the magnitude values of time samples within the second reference time T2 with respect to a particular sample time, but with the magnitude values of time samples within the first reference time T1 excluded.

If the transformation is performed for the reflection signals received at a particular sample time A in the lower diagram of FIG. 4, the magnitude values of sample times included in area D1 and area D2, which are within the first reference time T1, may be excluded from the computation of the mean value. The computation of the mean value may be performed for the magnitude value at sample time A and the magnitude values of the sample times included in area L and area R which are within the second reference time T2, and the computed mean value may be substituted as the new value at sample time A. The above procedure may be performed for all of the sample times. Consequently, the magnitude value of a particular sample time in the received reflection signals may be transformed into the mean value of sample times near the particular sample time, but with the magnitude values of relatively closer nearby sample times (within the first reference time) excluded from the computation of the mean value and the computation of the mean value performed for the magnitude values of relatively farther nearby sample times (i.e. sample times that are beyond the first reference time but within the second reference time). The magnitude value of the particular sample time may be replaced with the computed mean value.

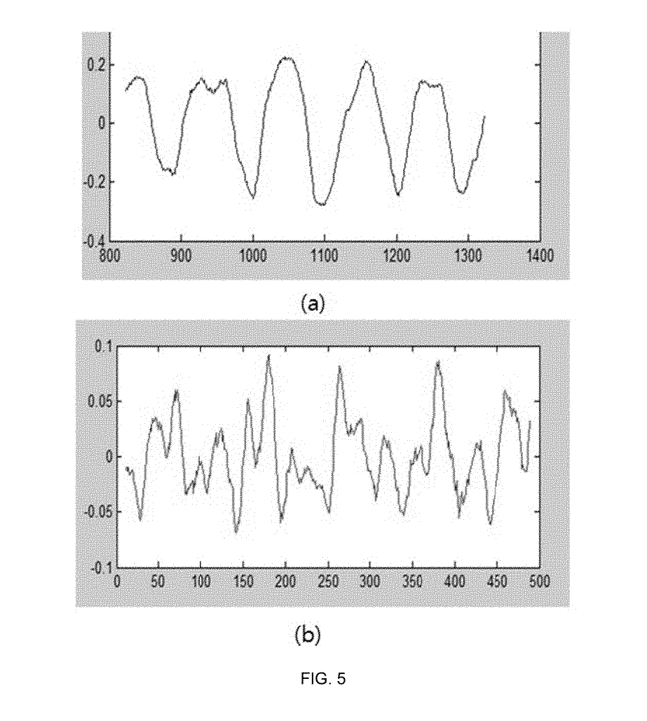

FIG. 5 shows graphs representing a waveform for the reflections signals of a UWB radar and a waveform for signals from which the respiration signals have been removed.

In FIG. 5, the upper diagram is a graph representing the waveform of the received reflection signals, and the lower diagram is a graph representing the waveform of the signals after the respiration signals have been removed from the received reflection signals.

Referring to the upper diagram of FIG. 5, the changes in the received signals are mainly due to the respiration signals having a low frequency, and the changes due to heartbeat signals are comparatively weak.

Referring to the lower diagram of FIG. 5, it can be seen that the heartbeat signals of a high frequency are observed more clearly when the respiration signals have been moved from the received reflection signals.

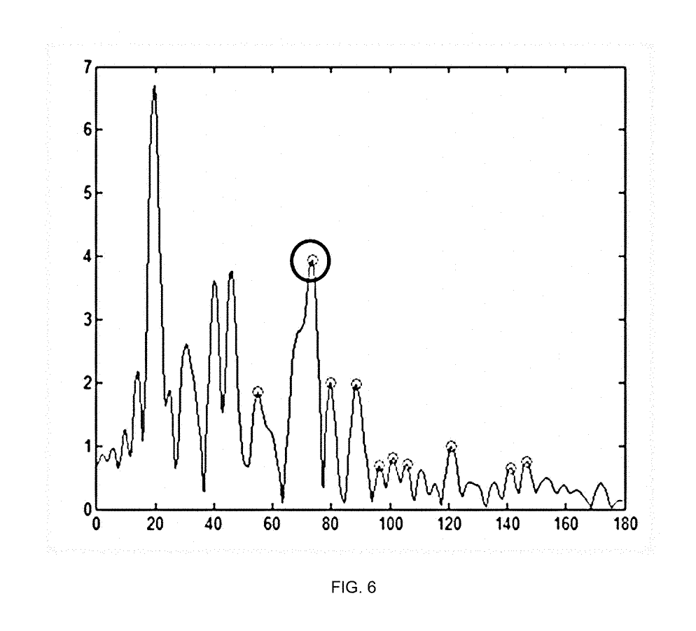

FIG. 6 is a graph illustrating the signals of the frequency domain when respiration signals have been removed from the received signals.

Referring to FIG. 6, the part marked with big circles is the parts corresponding to heartbeat signals. It can be seen from FIG. 6 that the heartbeat signals are observed considerably more clearly when the respiration signals have been removed from the received signals.

FIG. 7 is a flowchart illustrating a method of determining a heartbeat rate according to an embodiment of the invention.

The determining of the heartbeat rate can be performed using the received reflection signals as in the embodiment illustrated in FIG. 2 and can also be performed after the respiration signals are removed from the received reflection signals as in the embodiment illustrated in FIG. 3.

Referring to FIG. 7, a device for measuring biometric data according to an embodiment of the invention may analyze the frequency component signals of the signals obtained after removing the respiration signals from the or reflection signals or received signals reflected off the target patient (S700).

Here, a peak value refers to a value that is higher than its surrounding values, and multiple peak values can be detected. When the peak value detection is performed for the reflected received signals, the highest peak value from among the peak values may correspond to the respiratory rate. After the detection of the multiple peak values, a filtering may be performed that involves removing the harmonic components of the respiratory rate (S702). That is, the peak values corresponding to the harmonic components of the respiratory rate may be excluded from the multiple peak values.

The device for measuring biometric data according to an embodiment of the invention may determine the heartbeat rate by using at least one of the repetition frequencies and magnitudes of the peak values from among the frequencies for the peak values from which the harmonic components have been removed (S704). Here, the repetition frequencies and magnitudes for determining the heartbeat rate can be applied in various forms according to the algorithm.

For example, a device for measuring biometric data according to an embodiment of the invention can determine the heartbeat rate as the frequency corresponding to the maximum repetition frequency and maximum mean magnitude. That is, if peak values occur the most often at a particular frequency and the maximum mean magnitude is the highest at the frequency, then the frequency can be determined to be the heartbeat rate.

As described above, in cases where the procedures are repeated five times to generate five candidates, the peak values may be detected from within a predesignated frequency range. Then, ignoring the harmonic components for the maximum peak value, the heartbeat rate of the target patient can be identified by identifying which frequency has the most occurrences of peak values or which frequency has the highest magnitude.

Alternatively, a different weight can be assigned to each parameter, i.e. the repetition frequency and magnitude, and the frequency corresponding to the maximum repetition frequency and maximum mean magnitude can be determined as the heartbeat rate. Alternatively, since the maximum repetition frequency can appear at a first frequency while the maximum magnitude appears at a second frequency, different weights can be assigned to the repetition frequency and the magnitude, and the frequency corresponding to a parameter having the maximum value from among the weighted values can be determined to be the heartbeat rate. Alternatively, the heartbeat rate can be determined according to the maximum repetition frequency, but if there are more than one frequency having the same maximum repetition frequency, then the heartbeat rate can be determined by using the maximum magnitude additionally.

FIG. 8 represents the repetition frequencies and mean magnitudes of peak values detected during repetitions of the method of measuring biometric data illustrated in FIG. 7.

In FIG. 8, the horizontal axis represents per-minute frequency, and the vertical axis represents the repetition frequency and mean magnitude. As illustrated in FIG. 8, peak values occur the most often at about 72.64 per-minute frequency, and the mean magnitude is the highest at this frequency. Therefore, the heartbeat rate of the target patient can be determined to be about 72.64.

As described above, an embodiment of the invention can provide an accurate heartbeat rate of a target patient, in spite of the harmonic components of the frequency corresponding to the respiratory rate and in spite of noise components, by identifying which frequency has the most occurrences of peak values and using at least one of the magnitudes at the corresponding frequencies by way of removing the harmonic components or by way of repeated measurement.

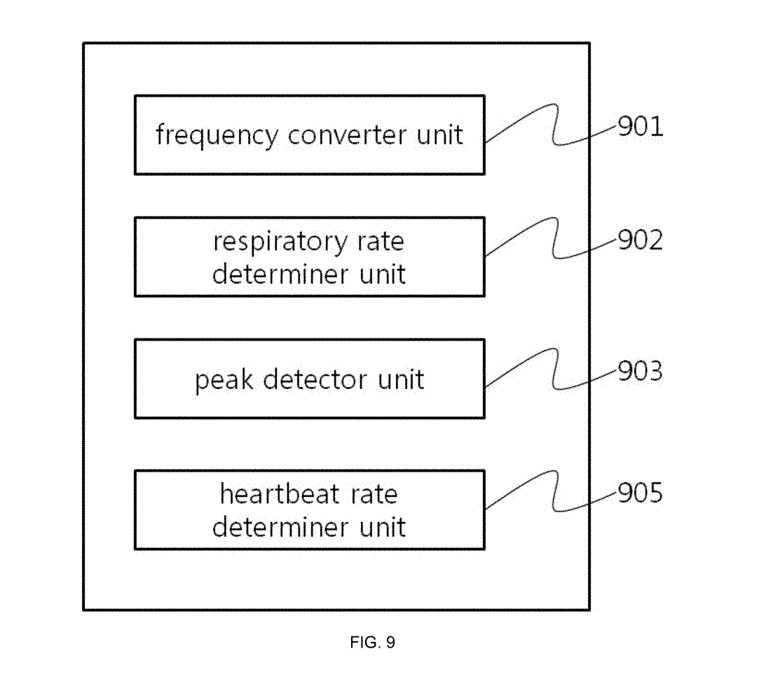

FIG. 9 illustrates a device for measuring biometric data according to an embodiment of the invention.

Referring to FIG. 9, the device for measuring biometric data according to an embodiment of the invention may include a frequency converter unit 901, a respiratory rate determiner unit 902, a peak detector unit 903, and a heartbeat rate determiner unit 905. The device for measuring biometric data according to an embodiment of the invention can be produced and sold separately from the UWB radar or be produced and sold together with the UWB radar.

The frequency converter unit 901 may convert the reflection signals received from the target patient into signals of a frequency domain. It is also possible to have the frequency converter unit 901 can convert the signals obtained by removing the respiration signals from the received reflection signals into signals of a frequency domain.

The respiratory rate determiner unit 902 may determine the respiratory rate by using the signals obtained by converting the received reflection signals into frequency-domain signals. The respiratory rate can be determined as the frequency corresponding to the largest peak value from among the converted frequency-domain signals.

The peak detector unit 903 may detect multiple peak values from the frequency signals converted by the frequency converter unit 901 and remove harmonic components of the signals associated with the respiratory rate. The detection of the peak values and the removal of the harmonic components can be performed for the signals obtained after converting the reflection signals into frequency-domain signals, or can also be performed for the signals frequency-domain converted signals obtained after removing the respiration signals from the reflection signals.

Here, the peak detector unit 903 can detect multiple peak values in a predesignated frequency range, where the predesignated frequency range can be designated according to the predicted heartbeat rate of the target patient.

The heartbeat rate determiner unit 905 may determine the heartbeat rate by using at least one of the repetition frequencies and magnitudes of the peak values, from among the frequencies for peak values detected according to a predesignated number of repetitions. That is, UWB radar signals can be emitted periodically for multiple time slots, and the frequency converter unit 901 and the peak detector unit 903 can analyze the frequency components of the reflection signals for each time slot and detect peak values. The peak values at each time slot may be extracted, and from among the frequencies for the detected peak values, the heartbeat rate can be determined, for example, as the frequency having the most occurrences of peak values or the frequency having the highest mean magnitude.

The technical details described above can be implemented in the form of program instructions that may be performed using various computer means and can be recorded on a computer-readable medium. Such a computer-readable medium can include program instructions, data files, data structures, etc., alone or in combination. The program instructions recorded on the medium can be designed and configured specifically for the present invention or can be a type of medium known to and used by the skilled person in the field of computer software. Examples of a computer-readable medium may include magnetic media such as hard disks, floppy disks, magnetic tapes, etc., optical media such as CD-ROM's, DVD's, etc., magneto-optical media such as floptical disks, etc., and hardware devices such as ROM, RAM, flash memory, etc. Examples of the program of instructions may include not only machine language codes produced by a compiler but also high-level language codes that can be executed by a computer through the use of an interpreter, etc. The hardware mentioned above can be made to operate as one or more software modules that perform the actions of the embodiments of the invention, and vice versa. While the present invention has been described above using particular examples, including specific elements, by way of limited embodiments and drawings, it is to be appreciated that these are provided merely to aid the overall understanding of the present invention, the present invention is not to be limited to the embodiments above, and various modifications and alterations can be made from the disclosures above by a person having ordinary skill in the technical field to which the present invention pertains. Therefore, the spirit of the present invention must not be limited to the embodiments described herein, and the scope of the present invention must be regarded as encompassing not only the claims set forth below, but also their equivalents and variations.

1-15. (canceled)

16. A method of measuring biometric data using a UWB radar, the method comprising:
(a) determining a respiratory rate by using reflection signals reflected off a target patient;
(b) extracting respiration signals from the reflection signals of time domain, removing the extracted respiration signals from the reflection signals and converting into signals of a frequency domain; and
(c) determining a heartbeat rate by detecting peak values from the frequency-domain signals converted in said step (b),
wherein extracting the respiration signals from the reflection signals of time domain in said step (b) comprises the steps of:
(b1) calculating a mean value of magnitude values of sample times within a second reference time from a particular sample time excluding sample times within a first reference time from the particular sample time, the first reference time being shorter than the second reference time and included in the second reference time; and
(b2) replacing a magnitude value of the particular sample time with the mean value,
wherein said steps (b1) and (b2) are performed for all sample times in a predesignated segment

17. The method of claim 16, wherein said step (c) comprises:
determining the heartbeat rate as a frequency corresponding to a peak value within a predesignated frequency range from among a plurality of detected peak values.

18. The method of claim 16, wherein said step (c) comprises:
removing harmonic components of the respiratory rate; and
determining the heartbeat rate by using the peak values having the harmonic component removed therefrom.

19. The method of claim 18, wherein said steps (a) through (c) are performed multiple times, and said step (c) comprises determining the heartbeat rate by using at least one of a repetition frequency of peak values and a magnitude of peak values.

20. The method of claim 18, wherein said removing of the harmonic components comprises:
detecting a plurality of peak values within a predesignated frequency range and removing peak values corresponding to harmonic components from among the detected peak values,
and the predesignated frequency range is determined based on a heartbeat rate of the target patient.

21. A device for measuring biometric data using a UWB radar, the device comprising:
a frequency converter unit configured to convert reflection signals reflected off a target patient into frequency-domain signals;
a respiratory rate determiner unit configured to determine a respiratory rate by using the frequency-domain signals for the reflection signals converted by the frequency converter unit;
a peak detector unit configured to detect a plurality of peak values from frequency-domain signals obtained by extracting respiration signals from the reflection signals of time domain and removing the extracted respiration signals from the reflection signals and to remove harmonic components of the determined respiratory rate; and
a heartbeat rate determiner unit configured to determine a heartbeat rate as one of the peak values remaining after removing the harmonic components,
wherein the respiration signals are extracted from the reflection signals of time domain by performing a procedure for all sample times within a predesignated segment, the procedure comprising:
calculating a mean value of magnitude values of sample times within a second reference time from a particular sample time excluding sample times within a first reference time from the particular sample time, the first reference time being shorter than the second reference time and included in the second reference time; and replacing a magnitude value of the particular sample time with the mean value.

22. The device of claim 21, wherein the heartbeat rate determiner unit determines the heartbeat rate as a frequency corresponding to a peak value within a predesignated frequency range from among the plurality of peak values detected.

23. The device of claim 21, wherein the heartbeat rate determiner unit determines the heartbeat rate using at least one of a repetition frequency of the peak values and a magnitude of the peak values for the reflection signals obtained over a plurality of repetitions.
